(19) United States
(12) Patent Application Publication
(10) Pub. No.: US 2023/0298739 A1
(43) Pub. Date: Sep. 21, 2023

(54) LOCALIZATION SYSTEM AND METHOD

(71) Applicant: Geonavo Positioning Systems, Inc., Hammonds Plains (CA)

(72) Inventors: Malcolm Williams, Ottawa (CA); Stewart Ian Hardie, Hammonds Plains (CA); Ioan Romulus Curticapean, Markham (CA); Benjamin Griffen Ryan Hudson, Waterloo (CA)

(73) Assignee: Geonavo Positioning Systems, Inc., Hammonds Plains, NS (CA)

(21) Appl. No.: 18/019,144

(22) PCT Filed: Aug. 5, 2021
(86) PCT No.: PCT/CA2021/051090

§ 371 Date: Feb. 1, 2023

Related U.S. Application Data
(60) Provisional application No. 63/061,438, filed on Aug. 5, 2020.

(51) Int. Cl. G16H 40/20; G01S 5/02

(52) U.S. Cl. CPC G16H 40/20; G01S 5/0244

(57) ABSTRACT

A system comprises a plurality of first computing devices, wherein each first computing device is wearable by a user or is affixed to an asset within an environment and configured to generate and transmit a message; and a plurality of second computing devices installed within the environment, wherein a portion of the plurality of second computing devices are configured to detect at least one of the plurality of first computing devices, receive messages transmitted by a detected first computing device, and timestamp the messages before transmitting the messages to a computing server system. The computing server system is configured to determine at least a location of the detected first computing device based at least upon the messages received from the portion of the plurality of second computing devices.

### CROSS REFERENCE TO RELATED APPLICATION

The application claims priority to U.S. Provisional Patent Application No. 63/061,438 filed on Aug. 5, 2020, entitled “LOCALIZATION SYSTEM AND METHOD,” the content of which is incorporated by reference herein in its entirety.

### FIELD OF TECHNOLOGY

The present disclosure generally relates to a localization system and method, and more particularly relates to real-time reliable localization technology for healthcare facilities to monitor and analyze behavioral patterns and activities that impact the health and safety of residents and staff.

### BACKGROUND

Hospitals, specialized care centers, nursing homes, or any other type of medical center, healthcare facilities these days are struggling to collect accurate, reliable resident and staff behavior data needed to drive higher levels of care quality and financial performance. For example, there have been increased government and agency regulations, compliance and litigation risks at these healthcare facilities. The major source of concern includes inaccurate, inconsistent care, and performance data, inefficient delivery of care, difficulties with staff health and safety, recruitment, and retention, and lack of standardization for care, quality, and financial performance.

Accordingly, there is a need for an accurate and real-time monitoring and analytics platform and technology for various healthcare facilities.

### SUMMARY

The present disclosure generally discloses a method and system for accurate and real-time monitoring and analysis of healthcare facility resident, staff and asset. In one embodiment, an Internet-of-Things (IoT) based computing platform is provided for collecting, monitoring, and reporting on residents and staff interactions and behaviors in real-time. Further, the present disclosure integrates residents electronic behavioral record (e.g., activities, interactions, incidents, meals, washroom visits, visit counts, dwell time, zone time, sleep time, pace and step count, etc.) and electronic health record to provide 360° view of residents' healthy and safety profiles. As a result, staff spend more time caring for residents and providers deliver better quality of care and performance outcomes. In sum, the disclosed method and system provide accurate, objective behavioral data to standardize delivery of safety, care quality and financial performance outcomes. Increased staff health, safety and satisfaction lead to improved staff engagement, recruitment and retention. Accordingly, healthcare facilities achieve reduced regulation, compliance, and litigation risks.

In accordance with aspects of the present disclosure, a system comprises a plurality of first computing devices, wherein each first computing device is wearable by a user or is affixed to an asset within an environment and configured to generate and transmit a message; and a plurality of second computing devices installed within the environment, wherein a portion of the plurality of second computing devices are configured to detect at least one of the plurality of first computing devices, receive messages transmitted by a detected first computing device, and timestamp the messages before transmitting the messages to a computing server system. The computing server system is configured to determine at least a location of the detected first computing device based at least upon the messages received from the portion of the plurality of second computing devices.

In one embodiment, the computing server system may be configured to obtain information relating to an environment in which the plurality of first and second computing devices are deployed. The information may comprise parameters indicating at least one of: locations of walls or physical obstructions of the environment that would impede movement wherein computing server system is configured to determine the location of the detected first computing device using at least the messages received from the portion of the plurality of second computing devices and the information.

In another embodiment, the computing server system may be configured to determine the location of the detected first computing device using at least time difference of arrival (TDOA) measurements from the portion of the plurality of second computing devices, and a single two way ranging (TWR) measurement from one of the plurality of second computing devices. The one of the plurality of second computing devices that performs the TWR measurement may be pre-selected based at least on an estimated position of the detected first computing device, an estimate of which ones of the plurality of second computing devices receive a next message from the detected first computing device, and a method for minimizing a measurement uncertainty. In yet another embodiment, the one of the plurality of second computing devices that performs the TWR measurement for the next message may be determined based at least on measurements received from the detected first computing device at a previous message, and a set of the plurality of second computing devices that are likely to receive from the detected first computing device at the next message, wherein, for each of the set of the plurality of second computing devices, an expected position error is calculated, and measurements from the previous message are used to estimate an expected variance of the TWR measurement, wherein the method is configured to select one of the plurality of the second computing devices with the minimum expected error and determine whether to replace a previously selected second computing device for performing the TWR measurement.

In an additional embodiment, the computing server system may be configured to determine the location of the detected first computing device using at least time difference of arrival (TDOA) measurements from at least one of the plurality of second computing devices, received signal strength indicator (RSSI) measurements from the plurality of second computing devices. The plurality of second computing devices may be calibrated to measure a propagation delay between itself and every other second computing device within a communication range using at least a single two way ranging (TWR) or double sided TWR (DS-TWR) methods. The plurality of second computing devices may be calibrated to estimate a quality of a communication channel between itself and every other second computing device within the communication range by measuring the propagation delay multiple times and calculating a standard deviation, fitting an arbitrary probability distribution, or other approximation from discrete data. In some embodiments, the plurality of second computing devices may be calibrated once during system installation, periodically, continuously, or as required when changes are detected in the system.

In yet another embodiment, the plurality of second computing devices may comprise master devices and slave devices. Each master device may selected from the plurality of second computing devices based at least on: maximizing an area formed by each cluster having a master device and multiple slave devices, having the best quality communication between each master device and the multiple slave devices, each second computing device has at least one master device, and minimizing a total number of required master devices. Each master device may be configured to periodically transmit a synchronization message, wherein the synchronization message includes at least information relating to a local clock of each master device at the time of transmission. Each slave device may be configured to receive the synchronization message and timestamp the synchronization messages upon reception.

In another embodiment, each of the plurality of second computing devices may be configured to determine a channel impulse response (CIR) for each signal received from each of the plurality of first computing devices, and the computing server system is configured to determine the location of the detected first computing device based at least on the CIR of each signal. The system may be deployed within a cloud-based network communication system.

In accordance with additional aspects of the present disclosure, a method comprises generating and transmitting one or more messages from each of a plurality of first computing devices wearable by a user or is affixed to an asset within an environment; installing a plurality of second computing devices within the environment; detecting, by a portion of the plurality of second computing devices, at least one of the plurality of first computing devices; receiving, by the portion of the plurality of second computing devices, messages transmitted by a detected first computing device; timestamping the messages by the portion of the plurality of second computing devices before transmitting the messages to a computing server system; and determining, by the computing server system, at least a location of the detected first computing device based at least upon the messages received from the portion of the plurality of second computing devices.

In accordance with additional aspects of the present disclosure, a non-transitory computer-readable medium storing computer codes executable by one or more processors of a system to: generate and transmit one or more messages from each of a plurality of first computing devices wearable by a user or is affixed to an asset within an environment, wherein a plurality of second computing devices are installed within the environment; detect, by a portion of the plurality of second computing devices, at least one of the plurality of first computing devices; receive, by the portion of the plurality of second computing devices, messages transmitted by a detected first computing device; timestamp the messages by the portion of the plurality of second computing devices before transmitting the messages to a computing server system; and determine, by the computing server system, at least a location of the detected first computing device based at least upon the messages received from the portion of the plurality of second computing devices.

The above simplified summary of example aspects serves to provide a basic understanding of the present disclosure. This summary is not an extensive overview of all contemplated aspects, and is intended to neither identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplary pointed out in the claims.

### BRIEF DESCRIPTION OF THE DRAWINGS

The accompanying drawings, which are incorporated into and constitute a part of this specification, illustrate one or more example aspects of the present disclosure and, together with the detailed description, serve to explain their principles and implementations.

FIG. 1 illustrates a localization system, according to aspects of the present disclosure;

FIG. 2 illustrates an example cloud-based system architecture, according to aspects of the present disclosure;

FIG. 3 illustrates an example electronic behavior/health record management server system, according to aspects of the present disclosure;

FIG. 4 illustrates a time difference of arrival (TDOA) between a single pair of computing devices, according to aspects of the present disclosure;

FIG. 5 illustrates a complete TDOA localization, according to aspects of the present disclosure;

FIG. 6 illustrates two clusters of master and slave computing devices, according to aspects of the present disclosure;

FIG. 7 illustrates an example clock synchronization messages, according to aspects of the present disclosure;

FIG. 8 illustrates an example localization showing mean squared error (MSE) contours, according to aspects of the present disclosure;

FIG. 9 illustrates an example particle filter in action, according to aspects of the present disclosure;

FIGS. 10(a) and 10(b) illustrate time of arrival range measurement and localization, according to aspects of the present disclosure;

FIG. 11 illustrates an example single sided two way range, according to aspects of the present disclosure;

FIG. 12 illustrates an example double sided two way range, according to aspects of the present disclosure;

FIG. 13 illustrates an optimized double sided two way range, according to aspects of the present disclosure;

FIGS. 14(a) and 14(b) illustrate a localization inside and outside of the polygon, respectively, according to aspects of the present disclosure;

FIG. 15 illustrates an example angle of arrival localization, according to aspects of the present disclosure;

FIG. 16 illustrates an ultra wide band (UWB) impulse with a 3494.4 MHz carrier superimposed, according to aspects of the present disclosure;

FIG. 17 illustrates an example UWB frame structure, according to aspects of the present disclosure;

FIGS. 18(a)-(c) illustrate three indoor environment channel scenarios;

FIG. 19 illustrates an indoor line of sight channel impulse response; and

FIG. 20 illustrates a non line of sight channel impulse response.

### DETAILED DESCRIPTION

Various aspects of invention will be described with reference to the drawings, wherein like reference numerals are used to refer to like elements throughout. In the following description, for purposes of explanation, numerous specific details are set forth in order to promote a thorough understanding of one or more aspects of the invention. It may be evident in some or all instances, however, that any aspects described below may be practiced without adopting the specific design details described below.

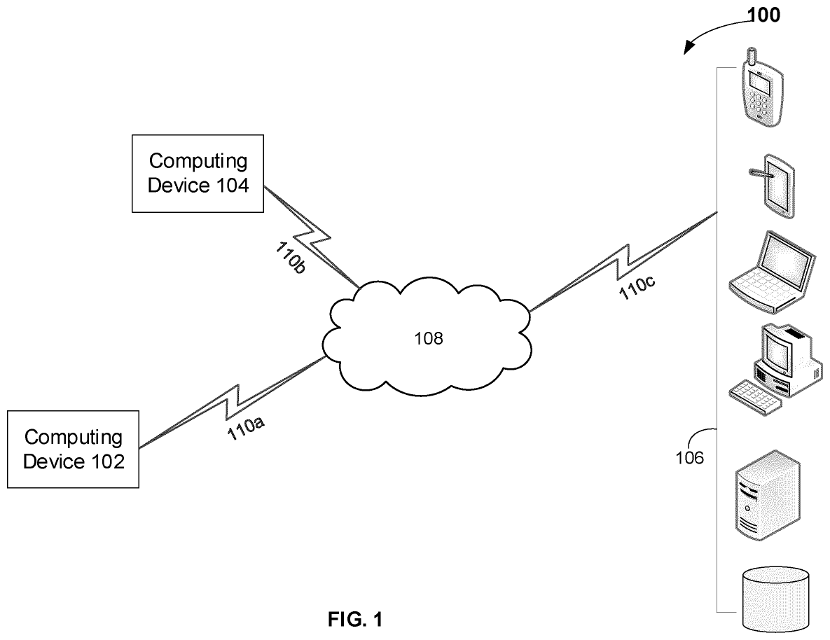

According to an embodiment, FIG. 1 generally illustrates a localization system 100 comprising at least one first computing device 102 which may be referred to as a “tag,” “tag device,” “bracelet” or “wearable,” and at least one second computing device 104 which may be referred to as an “anchor,” “beacon,” “beacon device,” or “anchor device.” The first computing device 102 may be mobile and/or body worn computing devices with their locations to be determined as described below. For example, device 102 may include any device that is capable of being worn or mounted at, on, in or in proximity to a body surface, such as a finger/toe, neck, wrist, arm, ankle, waist, chest, leg, ear, eye, head or other body part. Alternatively, device 102 may be configured to be part of a wristwatch, a head-mountable display, a headband, a pair of eyeglasses, jewelry (e.g., earrings, ring, bracelet, necklace, pin), a head cover such as a hat or cap, a belt, an earpiece, other clothing (e.g., a scarf), and/or other devices. In one embodiment, device 102 may be mounted directly to a portion of the body with an adhesive substrate, for example, in the form of a patch, or may be implanted in the body, such as in the skin or another organ. Generally, devices 102 may be small and comfortable so they may be worn at all times, and have a long battery life (e.g., 6-9 month battery life). Additionally, device 102 may be suitable to be mounted to a non-human asset to be tracked via adhesives, bolts, screws, permanent bonding, built into the asset, harnessed or otherwise affixed to the asset. Examples of assets may include beds, lifts, carts, equipment or other tangible objects. In one embodiment, device 102 may be configured to be powered by the asset or by an additional external power source such as a battery, solar panel or any suitable energy harvesting mechanism. When device 102 is in motion, it may localize, for example, once per second or based on a selected frequency. When device 102 is at rest, it may localize once every 60 seconds or based on a selected frequency.

One or more second computing devices 104 may be placed at selected locations during installation (e.g., on a wall or ceiling). Device 104 may be powered from 120V AC and configured to communicate with a computing server system 106, implemented locally or remotely. To localize, in one embodiment, computing device 102 may be configured to transmit an ultra-wide band (UWB) message, referred to as a “blink.” All computing devices 104 within range timestamp the message upon reception. The timestamp and the information contained within each message (e.g., battery life, firmware version, button status, unique identifier, etc.) may be transmitted to the computing server system 106. Other messages or measurements may be performed by device 102 and/or device 104 in conjunction with, or in place of the aforementioned blinks to localize device 102. Device 104 may independently perform some or all of the functions of device 102, in addition to the aforementioned functions. In another embodiment, system 100 may not need to rely on UWB technology, but rather uses any transmission protocol that is capable of digitally logging events or recording messages accurately (e.g., timestamping). That is, computing devices 102 and 104 may use any communication method and protocol where the transmission time, reception time and propagation velocity may be obtained and/or measured to a sufficient degree of accuracy. Furthermore, the information contained in each message (battery life, firmware version, button status, unique identifier, etc.) may be transmitted using a different medium than that which the underlying communication method or protocol may use for timestamping.

In one embodiment, computing devices 102, 104 and other devices of system 100 may be configured to communicate with the computing server system 106 via a communication network 108 using suitable network connections and protocols 110a, 110b, and 110c. A communication network (e.g., communication network 108) may refer to a geographically distributed collection of computing devices or data points interconnected by communication links and segments for transporting signals and data therebetween. A protocol (e.g., protocols 110a, 110b, and 110c) may refer to a set of rules defining how computing devices and networks may interact with each other, such as frame relay, internet protocol (IP), transmission control protocol (TCP), user datagram protocol (UDP), hypertext transfer protocol (HTTP). Many types of communication networks are available, ranging from local area networks (LANs), wide area networks (WANs), cellular networks, to overlay networks and software-defined networks (SDNs), a packet data network (e.g., the Internet), mobile telephone networks (e.g., cellular networks, such as 4G or 5G), Plain Old Telephone (POTS) networks, and wireless data networks (e.g., Institute of Electrical and Electronics Engineers (IEEE) 802.11 family of standards known as WiGig®, IEEE 802.16 family of standards known as WiMax®), IEEE 802.15.4 family of standards, a Long Term Evolution (LTE) family of standards, a Universal Mobile Telecommunications System (UMTS) family of standards, peer-to-peer (P2P) networks, virtual private networks (VPN), Bluetooth, Near Field Communication (NFC), or any other suitable network. Devices 102 and 104 may be configured to communicate in a peer to peer manner to replace, duplicate, supplement or extend the functionalities of communication network 108.

For example, communication network 108 may be a LAN configured to connect each of first and second computing devices 102, 104 and other devices deployed within a nursing home over dedicated private communications links. Communication network 108 may be a WAN configured to connect computing devices deployed within the nursing home and other geographically dispersed computing devices and networks over long-distance communications links, such as common carrier telephone lines, optical light paths, synchronous optical networks (SONET), or synchronous digital hierarchy (SDH) links. The Internet may be used to connect disparate devices and networks throughout the world, providing global communication among nodes (a node of an Internet has an IP address) on various networks. These nodes may communicate over the communication network 108 by exchanging discrete frames or packets of data according to protocols 110a-110c, such as TCP/IP. Communication network 108 may be further interconnected by an intermediate network node, such as a router and/or gateway device, to extend the effective size of each network.

In another aspect, system 100 may employ a cloud-based communication network 108 for providing computing services using shared resources. Cloud computing may generally include Internet-based computing in which computing resources are dynamically provisioned and allocated to each connected computing device or other devices on-demand, from a collection of resources available via the network or the cloud. Cloud computing resources may include any type of resource, such as computing, storage, and networking. For instance, resources may include service devices (firewalls, deep packet inspectors, traffic monitors, load balancers, etc.), compute/processing devices (servers, CPUs, GPUs, random access memory, caches, etc.), and storage devices (e.g., network attached storages, storage area network devices, hard disk drives, solid-state devices, etc.). In addition, such resources may be used to support virtual networks, virtual machines, databases, applications, etc.

Cloud computing resources accessible via communication network 108 may include a private cloud, a public cloud, and/or a hybrid cloud. For example, a private cloud may be a cloud infrastructure operated by an enterprise for use by the enterprise, while a public cloud may refer to a cloud infrastructure that provides services and resources over a network for public use. In a hybrid cloud computing environment which uses a mix of on-premises, private cloud and third-party, public cloud services with orchestration between the two platforms, data and applications may move between private and public clouds for greater flexibility and more deployment options.

In accordance with an aspect, computing server system 106 of the present disclosure may be cloud-based and may comprise at least one of personal computers, servers, server farms, laptops, tablets, mobile devices, smart phones, smart watches, fitness tracker devices, cellular devices, gaming devices, media players, network enabled printers, routers, wireless access points, network appliances, storage systems, gateway devices, smart home devices, virtual or augmented reality devices, one, a portion of a plurality, or a plurality of devices 102, one, a portion of a plurality, or a plurality devices 104, or any other suitable devices that are deployed in the same or different communication network of computing devices 102, 104. As will be described fully below, computing server system 106 may be configured to provide functionalities for any connected devices such as sharing data or provisioning resources among multiple client devices, or performing computations for each connected client device.

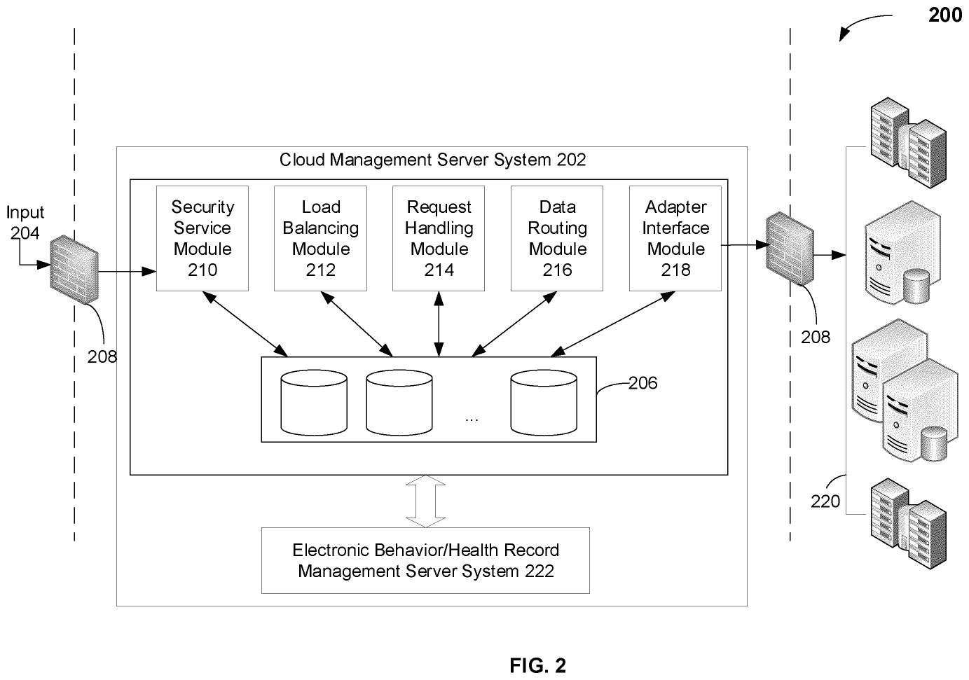

FIG. 2 shows an example architecture 200 of the system 100 of FIG. 1 using a cloud management computing server system 202 for exchanging information among different entities, according to aspects of the present disclosure. On a high level, the cloud management computing server system 202 facilitates on-demand delivery of compute power, database storage, software applications, and other IT resources through a cloud services platform via the Internet. The cloud management computing server system 202 may include multiple cloud servers concurrently running on a hypervisor to control the capacity of underlying operating systems and allocate processor cycles, memory space, network bandwidth and so on. Input 204 (e.g., data and messages) to the cloud server system 202 may be obtained from devices 102, 104, or other data sources or computing devices connected therewith. In one embodiment, input 204 may include cloud service invocation messages, result messages, request messages, or any messages communicated among different cloud computing devices. For example, a message may include a message type (e.g., a type value from a set of shared type constants), a unique identifier (e.g., an identifier used to correlate this message with one or more other messages), priority information to support for priority based message queues, timeout, sensitivity indicator to support message data isolation, message source (e.g., a uniform resource identifier (URL) of a sender), a message destination (e.g., a URL that uniquely identifies the destination), a request context (e.g., request information from a dispatcher), and/or a message payload. The payload may have different attributes depending upon the type of message that is being sent, such as parameter data and result data.

The cloud management computing server system 202 may be configured to operate as a secure intermediary computing environment for real time or near real time data collection, storage, and analysis in connection with the use of devices 102, 104. For example, server system 202 may implement techniques to facilitate communications among various mobile computing devices and cloud computing entities (cloud datacenters, cloud web servers, cloud application servers, cloud database servers, cloud storage devices) despite their incompatibilities in communication, such as differences between formats or communication protocols. In certain embodiment, server system 202 may be configured to translate communication protocols among different computing devices.

Server system 202 may be implemented using hardware, software, firmware, or combinations thereof. For example, the cloud management server system 202 may include one or more computing devices, such as a server computer, one or more memory storage data repositories 206, one or more processors, and operate with different kinds of operating systems. Each memory storage device may implement one or more databases (e.g., a document database, a relational database, or other type of database), one or more file stores, one or more file systems, or combinations thereof, and may include instructions stored thereon which, when executed by the processor(s), cause the processor(s) to implement one or more operations disclosed herein.

Data repositories 206 may be accessible by various modules 210-218. For example, one of the data repository 206 may store all the metadata (e.g., run-time and design-time data, each having their own requirements on availability and performance) associated with the server system 202. A tenant or subscriber (e.g., device 102 or 104) of the server system 202 may have any number of applications installed thereon. Each application may be versioned and have at least one versioned resource application programming interface (API), and corresponding versioned service. The data repository may store one or more callable interfaces, which may be invoked by device 102, 104. The callable interface may be implemented to translate between a one format, protocol, or architectural style for communication and another format, protocol, or architectural style for communication. Further, another data repository 206 may be used to store information about processing occurring in the cloud management server system 202, such as messages communicated via the server system 202 and log information. Additional data repositories 206 may be configured to store logging and analytics data captured during processing in the cloud management server system 202. Depending on the demand of computing devices seeking to communicate with backend cloud resources 220, the cloud management server system 202 may be configured to handle surges and temporary periods of higher than normal traffic between each mobile computing device and other cloud computing devices. For example, the cloud management server system 202 may include elements that support scalability such that components may be added or replaced to satisfy demand in communication.

Input 204 (e.g., a request for cloud service) may be communicated between device 102 or 104 and the cloud management server system 202 via one or more callable interfaces, e.g., APIs. The cloud management server system 202 may be protected by one or more firewalls 208 to provide a secure environment to process requests from various computing devices. For example, firewalls 208 may permit communication of messages between the cloud management server system 202 and each device 102 and/or 104. Such messages (e.g., SPDY messages, hypertext transfer protocol (HTTP) messages or representational state transfer (REST) messages) may conform to a communication protocol (e.g., SPDY, HTTP, or REST). Input 204 that is received through the firewall 208 may be processed first by security service module 210 which is configured to manage security authentication for a user associated with a service request by at least restricting access to only those who have the required credentials to certain patient medical data. In one embodiment, security authentication may be determined for a request, a session, a user, a device, other criterion related to the user, or combinations thereof. Security authentication may be performed for each request that is received or based on a previous verification of a request. Security authentication may be determined for a user or a device, such that requests to different cloud services 220 may be authenticated based on a single verification of security.

Upon determining security authentication, the cloud management server system 202 may use the load balancing module 212 to detect to which cloud service 220 the received request is directed, and use a request handling module 214 to transmit each service request to an appropriate cloud service 220. A request may be routed to an appropriate service 220 upon dispatch, or to another module of the cloud management server system 202. The request handling module 214 may resolve a request to determine its destination based on a location (e.g., a URL of the request). The request handling module 214 may parse a request's header to extract one or more of the following information: tenant identifier, service identifier, application name, application version, request resource, operation and parameters, etc. The request handling module 214 may use the parsed information to perform a lookup in data repositories 206 and retrieve corresponding application metadata. The request handling module 214 may determine the target service based on the requested resource and the mappings in the stored metadata. Via formatting the request and any other necessary information, the request handling module 214 may place the input message on data routing module 216 for further processing, or on a queue and await the corresponding response. The request handling module 214 may process responses received from the data routing module 216 and return a response to, e.g., the device 102 and/or 104.

The data routing module 216 may manage delivery of messages to destinations registered with itself The data routing module 216 may operate as a central system for managing communications in cloud services 220, such that additional centralized services (additional authorization, debugging, etc.) may be plugged in as necessary. Data captured by the data routing module 216 may be stored in the data repositories 206.

The data routing module 216 may route messages to one or more destinations 220 directly, or with the aid of an adapter interface module 218 by translating or converting a message to a protocol supported by a receiving cloud device 220. The adapter interface module 218 may establish separate communication connections with each of cloud resources 220.

In accordance with aspects of the present disclosure, the cloud management server system 202 may implement an electronic behavior/health record management server system 222 for obtaining real time data from devices 102, 104, and/or other data sources, such as stored historical data, conducting data capture, storage, analysis, search, sharing, transferring, querying, and updating of the obtained data using proprietary algorithms, and providing feedback to a patient in a real time, near real time, daily, monthly, or at a user requested interval to enhance therapeutic outcome. The data may also be used to predict behavior or health outcomes to enhance care.

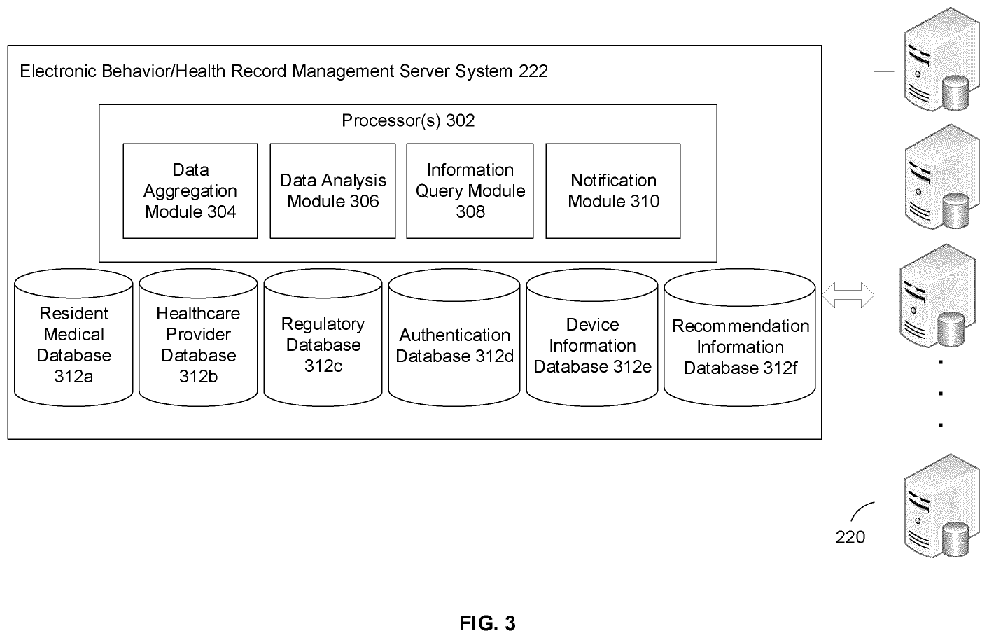

Referring to FIG. 3, the electronic behavior/health record management server system 222 may include one or more processors 302 communicatively coupled to a plurality of information databases 312a-312f. The server system 222 may also be configured to access the plurality of servers and cloud-implemented processing, memory, and data resources 220 connected with the underlying management server system 202 shown in FIG. 2. Thus, as information dynamically changes, the electronic behavior/health record management server system 222 may be configured to scale with additional processing resources, server resources, data storage resources, and data management resources.

Databases 312a-312f may include database(s), database management system(s), server(s) to facilitate management, provision, transfer, and analysis of various patient healthcare information. For example, database 312a may retain any confidential or publicly available medical information of a particular resident of a healthcare facility collected from various data sources by a data aggregation module 304. Example medical information may include, but not limited to, any information on resident's health conditions, medical conditions, characterizations, assessments, test results, biographical or demographical information, prescription information, immunization records, care services provided, insurance policy information, coverage/benefits guidelines/rules for care services, healthcare plans, explanations of benefits, and the like.

Further, information database 312b may retain any relevant information related to various healthcare or services providers (e.g., physicians, nurses, pharmacists, or labs). The provider information may include, but not limited to, provider identification information, provider location, amenities offered by providers, provider schedule information, technology offered by providers, preventive/curative/palliative/other care service offerings information, in-network/preferred provider information, advertising information, provider billing information, reviews of providers, provider feedback, and the like.

Moreover, information repository 312c may retain relevant health information regulatory rules. The regulation information may include, but not limited to, regulations issued by a government authority, such as the Departments of Health and Human Services, Labor, and Treasury that require insurance plans/issuers to cover certain preventive services delivered by in-network providers without any cost-sharing. The regulation information may also include information relating to Health Insurance Portability and Accountability Act of 1996 (HIPAA) regulatory policies, procedures, and guidelines for controlling and maintaining privacy/security of confidential health information.

As resident's medical information and regulatory information exchanging among different entities, database 312d may retain relevant authentication information to facilitate privacy and security for data accessing and transferring. For example, the authentication information may restrict or grant access to certain patient's confidential health and/or other system-provided features via various communication interfaces.

In accordance with aspects of the present disclosure, a device information database 312e may be configured to obtain and store real-time data from devices 102 and 104, determine relativeness of data received from devices 102, 104 and from other sources, and exchange information with other modules or computing devices via appropriate interfaces.

Moreover, a recommendation information database 312f may be configured to encode knowledge extracted from various sources of medical expertise in a number of rule sets. Knowledge here refers to a representation that resembles the way experts tend to express most of their problem solving techniques. Each rule set may comprise a set of condition-action rules. Each single rule may describe a situation in which a certain action needs to take place. The recommendation information may include text, audio, video, and other rich media explanations to staff or health providers at various healthcare facilities.

To generate customized recommendations in connection with the data received from devices 102 and 104, the processor(s) 302 may control and execute a number of modules including a data aggregation module 304, data analysis module 306, information query module 308, and notification module 310.

More specifically, the data aggregation module 304 may be configured to utilize one or more communication interfaces to access one or more of the databases 312a-312f, and/or the other data source(s) 220 through appropriate network connections, determine a degree of reliability, consistency, comprehensiveness, thoroughness, and accuracy of obtained information corresponding to a specific patient, and aggregate the obtained information using appropriate data structures for further data storage or processing. For example, the data aggregation module 304 may access manifold sets of confidential health information that corresponds to a specific resident from various data sources. Such date aggregation may include organizing, categorizing, qualifying, and comparing different sets of information;

detecting, identifying, and handling errors and discrepancies. Thus, the data aggregation module 304 may be configured to store the aggregated patient information in one or more of the databases 312a-312f.

Moreover, the data aggregation module 304 may acquire and store authentication information in the authentication database 312d. For example, a user, who may be a resident, a legal representative, or a healthcare provider, may use an interface of a computing device to seek access to resident's confidential health information with a set of credentials. The authentication information, which may be of any suitable form and content, may be retrieved and used to check the credentials provided. Pursuant to authentication, the user may be granted access to at least a portion of the stored information in databases 312a-312f corresponding to the identified patient.

In one aspect, the data aggregation module 304 may be linked to a remote server 220 that provides updates on information changes in databases 312a-312f corresponding to the identified patient, periodically crawl for updates and changes, or may otherwise receive notice of information changes from other data sources. Thereafter, the data aggregation module 304 may process the changes to identify the content and scope of the changes, and potential ramifications. For example, the data aggregation module 304 may correlate the changes with stored resident's information, determine that the changes affect prior recommendations, and determine that the changes translate to greater or lesser thresholds for various personalized, up-to-date recommendations to support, e.g., a resident's medication regime. As will be described fully below, to facilitate the dynamic nature of the resident's electronic behavior/health record management server system 222, the data analysis module 306 may utilize information stored in databases 312a-312f for situation analysis and implement a number of rules to generate recommendations. For example, using knowledge representation and reasoning, the information stored in databases 312a-312f is semantically linked and formally structured by the data aggregation module 304, such that the data analysis module 306 may be configured to analyze new information additions in databases 312a-312f and changes in rules from experts and automatic learning, perform information changes, and propagate the changes to relevant modules. After each change implementation, the data analysis module 306 may log and maintain these changes for later audit purposes including change recovery and for understanding the evolution history of the rules.

To ensure accuracy and relevancy of each recommendation, the data analysis module 306 may assess information relating to a recommendation rule set and assign a weight to the information. For example, missing information may have a lower score than non-missing information. Information may be weighted according to its data source. That is, information obtained from a healthcare provider may be weighted higher or lower relative to information provided by a resident; information collected from devices 102, 104 may be considered more reliable than corresponding or conflicting information self-reported by the resident. Based on the assigned weight, one or more follow-up questions prompting for further information or clarifying information may be generated by an information query module 308.

The information query module 308 may be configured to handle feedback received from e.g., staff or health providers in order to search, retrieve, modify, or facilitate transfer of particular information among different modules and information repositories.

Notification module 310 may be configured to generate and deliver an alert based on user preferences through one or more channels, detect user responses, and take further actions based on the responses. Some alert channels may include known communication resources, either one-way or two-way. Examples include SMS, Twitter, push notifications, and Google Cloud Messaging.

In another embodiment, the electronic behavior/health record management server system 222 may provide access to social services which provide basic integration with many of the popular social media sites such as Facebook, Twitter, etc. These services may allow for third party authentication using the user's credentials from those sites as well as access to their services. Examples include sending a tweet or updating a user's status and sharing experience and comments.

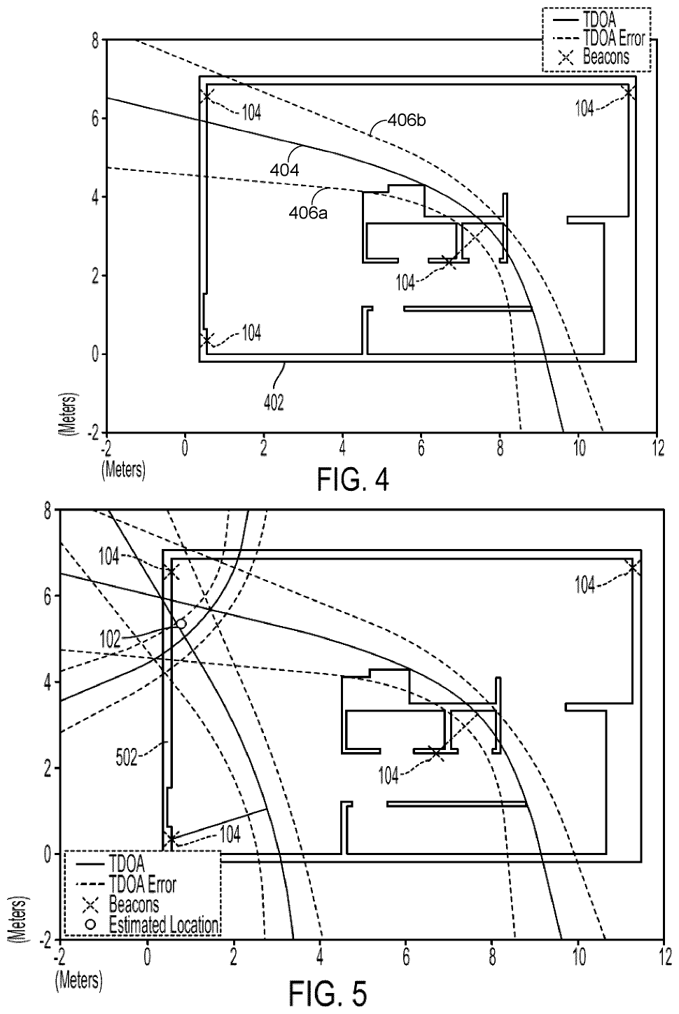

In accordance with aspects of the present disclosure, a time difference of arrival (TDOA) of each message may be computed by computing server system 106 for each pair of computing devices 104. FIG. 4 shows an example with a floorplan 402, a TDOA measurement 404 carried out by a number of computing devices 104, and the measurement uncertainty 406a and 406b. Specifically, line 404 is a hyperbola that represents all possible locations of a computing device 102 that would have generated this time difference within the floorplan 402. Lines 406a and 406b represent the error bounds on the measurement 404. The state of computing device 102 may be estimated using a combination of various parameters indicative of environment, measurement and prior state information. For example, environment information may include a floorplan (e.g., floorplan 402) showing the location of walls or other obstacles, knowledge of the construction materials, knowledge about the tags use case, who is using it, usage patterns in the past, models of the environment or any other prior knowledge about the environment. State may include position, velocity, acceleration, orientation, wakefulness of one or more users, which room a specific user is in, what activity the user is performing, or other information that may be estimated or determined. The state estimate may also include confidence measurements for the estimated values. Measurement information may include the TDOA between each pair of computing devices 104, range from each device 104, signal strength received by each device 104 (e.g., received signal strength indicator (RSSI) measurements of each signal), angle of arrival at each device 104, barometric pressure at each device 104, or measurements from sensors onboard each computing device 102 such as accelerometer, gyroscope, magnetometer, pedometer, biometrics, pressure, strength of nearby signals or other data that may be measured and facilitates the state estimation, along with any associated uncertainty measurements. In one embodiment, a number of computing devices 104 may be installed prior to localization and the placement of each device 104 may be determined by desired coverage density, outline of coverage area, ease of installation, target accuracy and/or simulation.

In one embodiment, each computing device 102 may include an accelerometer and gyroscope and optionally a magnetometer (referred to as an Inertial Measurement Unit/circuitry (IMU)). Each of these sensors may measure in up to three axes and obtained data may be combined with other measurements to improve the quality of the localization of a corresponding computing device 102. The IMU may operate at a higher update rate (e.g., 25-50 Hz) to provide more data points between updates. To conserve battery, the data may be buffered locally on the IMU, and is then read in one large operation, or a number of smaller operations by a processor of each computing device 102 prior to each transmission (e.g., once per second). This allows the processor to remain in a low power sleep mode between transitions, maximizing battery life. The raw IMU data may be included in the transition, may be compressed and transmitted, may be operated on locally and transmitted, or some combination of thereof The compression may be lossless (the original information may be perfectly reconstructed from the compressed information) or lossy (the reconstructed information may be an approximation of the original information with some loss). In one embodiment, the IMU data may be operated on by computing device 102 to estimate the pose of computing device 102 using estimation techniques such as a Kalman filter or a Magdgwick filter. All of the resulting pose data (x, y, z, roll, pitch, yaw) may be included in the transmission, or a subset such as only (x, y, z) may be included in the transmission. To reduce the number of bytes to be transmitted, data points with little change may be left out, only a delta may be transmitted, or the parameters of a curve that approximates the data may be transmitted, such as an nth order polynomial, single or multiple splines, wavelets or other. The processed IMU data may be combined with other time of flight measurements such as TDOA, TWR or phase difference of arrival (PDOA) using a Kalman or other filter to improve the accuracy of the localization estimate. The processed IMU data may also be used to provide a better motion model to a Particle filter or other filter to guide the updates of the particles using the velocities estimated from the IMU data to provide a better location estimate and/or reduce the number of particles required.

Since the IMU data is also at a higher frequency than other measurements, it may be used to interpolate movement of computing device 102 in between, e.g., TDOA, updates. In one embodiment, a rolling average pose change is used to estimate the wakefulness of a user wearing computing device 102. In one embodiment, when the pose change is below a threshold and other data such as location (if the user is near a bed, chair or other resting area, or a location that they are routinely asleep) and/or time of day (e.g., at night, when indicated that the user is expected to be asleep, or when historically has been asleep), the user may be estimated to be resting. The pose change threshold may be determined globally, on a per user class basis (e.g., categorized by age group, expected healthiness, or role), or on a per user basis. In another embodiment, the pose data may be used to estimate the gait of the user, which then may be used to estimate health deterioration, or interactions with medication. The gait may be classified based on extracted parameters such as step length, step time, vertical and horizontal deviations between steps, and/or step to step variability. In another embodiment, the processed IMU data may be used to detect a sudden fall event. Fall events may include any rapid or slow impacts with the floor or other movable or unmovable object, such as tripping and hitting the ground while walking, sliding off a bed while entering or exiting, fainting or developing a weakness. The detection of a fall event may be carried out on computing device 102, computing device 104 and/or computing server system 106. If a potential fall event is detected, computing device 102 and/or computing device 104 may enter a high power consumption mode where more data is collected to facilitate identifying the potential event. In one embodiment, computing device 102 may be configured to increase the rate at which IMU data is gathered, decrease the compression ratio of the IMU data, begin to gather, or increase the rate at which data is gathered with another sensor such as a barometer, increase the rate at which TDOA measurements are transmitted, and/or transmit different types of messages such as more TWR exchanges.

In accordance with aspects of the present disclosure, a TDOA of each message may be computed by computing server system 106 for each pair of computing devices 104. Pairs of computing devices 104 may be selected from a set of computing devices 104. The set of computing devices 104 may be defined as all computing devices 104 that received sufficient measurements to provide useful input to the localization event. In one embodiment, pairs may be generated by selecting one computing device 104 with the lowest measurement uncertainty as a reference beacon. The reference beacon may be paired with all remaining computing devices 104, such that a set of n computing devices 104 would result in n−1 pairs. In another embodiment, beacon pairs may also be selected as all possible combinations without repetition in the set, such that a set of n beacons would result in (n!)/(2! *(n−2)!) pairs.

With multiple beacon pairs, the location of a computing device 102 that generates and transmits the message may be determined. Referring to FIG. 5, in one embodiment, a number of computing devices 104 positioned within a location or environment 502 may be time-synchronized in order to calculate the relative TDOA. Statistical inference may be used to combine multiple measurements, environment and prior state information to estimate the state of a computing device 102. Any suitable statistical inference methods may be used for the state estimate including machine learning, Bayesian estimation, or any approximate Bayesian computation such as Maximum Likelihood Estimators, Kalman Filters, or Sequential Monte Carlo (Particle) Filters. FIG. 5 shows multiple TDOA measurements and the position estimate portion of the device 102's state.

Each computing device 104 may have a local clock that may be imperfect and drift over time. The TDOA localization algorithm of the present disclosure computes the TDOA between two different devices 104 by taking the clock drift of each device into consideration. To solve the problem, in one embodiment, all devices 104 may be wired to a central clock source. In a preferred embodiment, a method of wirelessly synchronizing clocks of all devices 104 may be contemplated. To accomplish this, a number of devices 104 may be selected as “masters.” These master devices 104 may be configured to periodically transmit synchronization messages, which may contain the value of their local clock at the time of transmit. The “slave” devices 104 within range of the master device may be configured to receive the messages and timestamp the reception. This information may be transmitted to the server system 106, and used to translate messages received by the slave devices 104 into the master time-base so a TDOA may be calculated. It should be appreciated that master/slave, or primary/secondary, or client/server, or controller/peripheral may refer to a model of asymmetric communication or control where one device or process (the “master” or “master device”) may be configured to control one or more other devices or processes (the “slaves” or “slave device”) and serve as their communication hub. For example, in some implementations, a master may be selected from a group of eligible devices, with the other devices acting in the role of slaves. In another embodiment, each slave device may perform the translation to all master time bases locally, before sending the resulting information to the server system 106.

In one embodiment, the rate at which master devices generate and transmit synchronization messages may be predetermined and fixed. In another embodiment, the rate may be dynamically adjusted. A shorter sync period will allow for better synchronization, but will consume more channel bandwidth. The average channel utilization may be periodically estimated by recording how many messages are received over a given duration and knowing how long each message is. Depending on the channel access method, such as ALOHA random access, slotted ALOHA, or Time Division Multiple Access (TDMA) a decision may be made to increase or decrease the synchronization rate. In one preferred embodiment, ALOHA may be used as the channel access method such that a probability of message collision may be calculated using the bandwidth utilization. Given a maximum allowable probability of message collision, the sync rate may be selected. The sync rate may be selected such that it is not a multiple of the computing device 102's transmission rate to prevent multiple sequential collisions with a computing device 102.

In one embodiment, a master device that is a slave to one or more other master devices may dynamically adjust the start time of its synchronization message to be spaced further apart from other synchronization messages. This prevents collisions between two master devices and increases the likelihood that a transmission from a computing device 102 will be close to a synchronization message. The closer a transmission from a computing device 102 is to a synchronization message, the less time there is for the clock to drift on computing device 104, meaning the calculated time may be more accurate. In one embodiment, the synchronization start time may be adjusted in small increments either earlier or later to move away from nearby synchronization messages. In another embodiment, the start time may be selected as the center of the expected largest free block of airtime in the next sync period. The expected largest free block of airtime may be estimated based on arrival times of synchronization messages from the last n sync periods. To prevent multiple master devices from adjusting their sync period into the same timeslot, a random chance of not updating the start of the synchronization message may be implemented, or the master devices exchange messages with other nearby master devices signaling their intent.

The master devices 104 may be selected from all installed devices 104 within an environment (e.g., a healthcare facility) to maximize localization accuracy. The following criteria may facilitate the selection:
1. Having the most number of slave devices, so each cluster may cover the largest area possible.
2. Best quality communication with the slave devices, i.e., clear line of sight to each slave device so the timestamps have a high quality.
3. Every computing device 104 within the healthcare facility has at least one master device.
   The masters may be selected to meet the above criteria using an optimization method, or using a heuristic to arrive at an approximate solution. Here, a heuristic may refer to an approach to problem solving or self-discovery that employs a practical method that may be sufficient for reaching an approximation. For example, an artificial intelligence system may be used to search a solution space. The quality of the communication with the slave devices may be measured in a number of different ways, one of which may be a calibration procedure. In one embodiment, the masters may be selected by building a number of possible solutions, evaluating the expected error of each solution, and selecting the one with the minimum error. One embodiment may include setting all computing devices 104 to masters, randomly setting one as a slave, and then determining if the criteria are still satisfied. In one embodiment, example criteria may include: all computing devices 104 must have at least one master, all masters must have at least a minimum number of slaves (e.g., 3), and there must be a target median number of masters per slave (e.g., 2). If the criteria are still met, a new computing device 104 may be selected at random and set as a slave, and the criteria are tested again. If the criteria are not met, one may set the computing device 104 back to a master and increment a “failure” counter. When the failure counter has reached a target threshold, for example 1000, the set of devices 104 that are still marked as being masters are a potential solution. The expected error may be calculated by determining the median expected steady state Kalman filter covariance throughout the venue. The input covariances for the Kalman filter may be estimated from the calibration data, and/or may be estimated from knowledge of the facilities construction materials and their effect on the signal properties. In another embodiment an initial guess at an optimal the master layout may be determined using a heuristic such as K-mean or K-medoids to arrive at an approximate solution that is then refined with the above process. In another embodiment, the approximate solution may be improved by identifying high error locations with a clustering algorithm such as density based spatial clustering (DBSCAN) and refining the selection in those specific areas. In another embodiment, if a computing device 104 detects that it has an insufficient number of masters, it may promote itself to master or it may request from computing server system 106 that a nearby computing device 104 be promoted to master.

In one embodiment, a calibration procedure may be performed once during system installation, periodically (e.g., nightly), continuously, as required when changes are detected in the system etc. For example, the calibration procedure may measure the propagation delay between every possible anchor pair. The propagation delay may be measured with TWR, DS-TWR, or any other suitable method. The propagation delay may be measured many times in succession, for example 100. Statistics may be computed from the gathered propagation delays such as mean and standard deviation which may be used to estimate the quality of the communication with the slave devices. Other statistics may be computed by fitting different probability distributions, or with other probability density approximation methods. The quality of the communication directly impacts the quality of the clock synchronization and hence the resulting TDOA. The quality information is used in multiple situations, including weighting TDOA measurements from a low quality pair less in the final localization to improve accuracy, estimate building materials, and optimally select master devices from the plurality of computing devices 104.

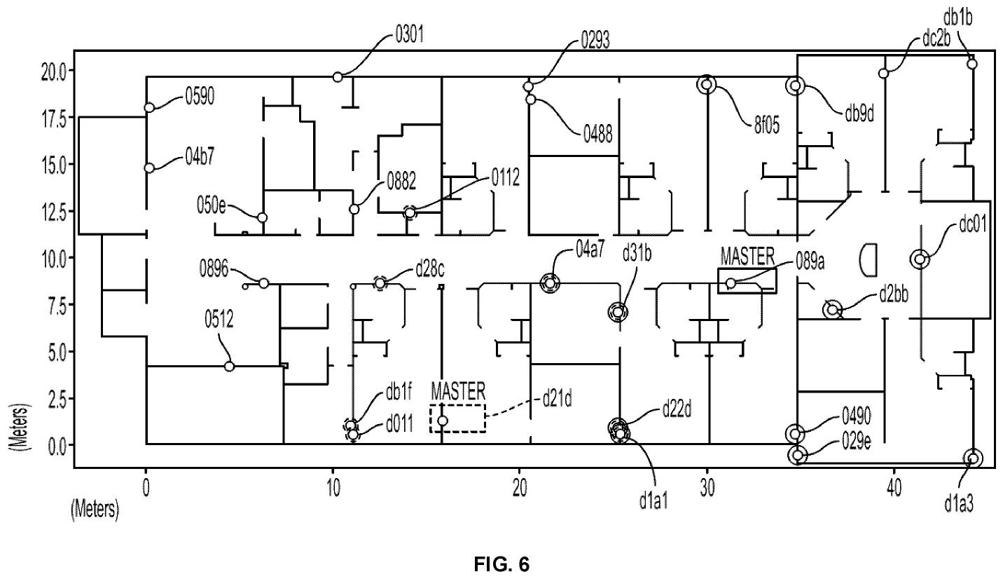

FIG. 6 shows an example layout with two clusters, where 089a and d21d are masters, and their slave devices are (8f05, db9d, dc01, d2bb, 0490, 029e, d22d, d1a1, d31b, and 07a7) and (0112, d28c, db1f, d011, 04a7, d31b, d22d, and d1a1), respectively. It should be appreciated that, depending on the masters selected, a device 104 may be a slave device to multiple overlapping clusters (e.g., 04a7, d31b, d22d, and d1a1). In this case, a TDOA between the slave and each master may be calculated. The timestamp of a message transmitted by a computing device 102 may be translated to each master time base and multiple TDOAs may be calculated. Further, a master device for one cluster may be a slave to another cluster. Although having a single master that is used for synchronization across all slave devices is much simpler, the ability to support multiple overlapping clusters is a key requirement to providing accurate and continuous coverage over a large area of an overall environment. The selection of which subset of the plurality of computing devices 104 as masters may be much more complicated when multiple need to be selected. The selection may have a very large impact on the quality of the synchronization, and hence location accuracy.

In another embodiment, each master's time base may optionally be converted to a global time base. To do this, every computing device 104 pair that has received synchronization messages from multiple masters computes a single TDOA that is representative of all the information contained in each TDOA calculated for each master time base. If the TDOA is modelled as a normal distribution, the mean and the variance may be averaged like any other normal distribution. A similar principle may be applied for other probability distributions.

In accordance with an aspect of the present disclosure, computing server system 106 may implement a localization engine (not shown) for all data processing. In one embodiment, messages generated and transmitted by computing devices 102 may be time synchronized. Thereafter, all messages for each localization may be collected and a localization may be computed by the localization engine. The updated tag state which may contain information such as position, velocity, acceleration, orientation, wakefulness of the user, which activity the user is doing, confidence associated with any of the previously mentioned values or any other value to be estimated of the localization may be transmitted to a front end that consumes the output of the localization engine to perform one or more tasks (e.g., display, storage, notification triggering, or other applications).

Clock synchronization may be carried out for each master/slave device pair. The process waits on messages from computing devices 104. When a message is received from a computing device 102, it is stored. When a master sync is received, all waiting messages may be translated into the master's time base and sent to a tag collector computing device of the localization engine. Linear interpolation may be used to translate the timestamp of the reception of a message from the tag into its master time base. Any suitable methods other than linear interpolation may be used.

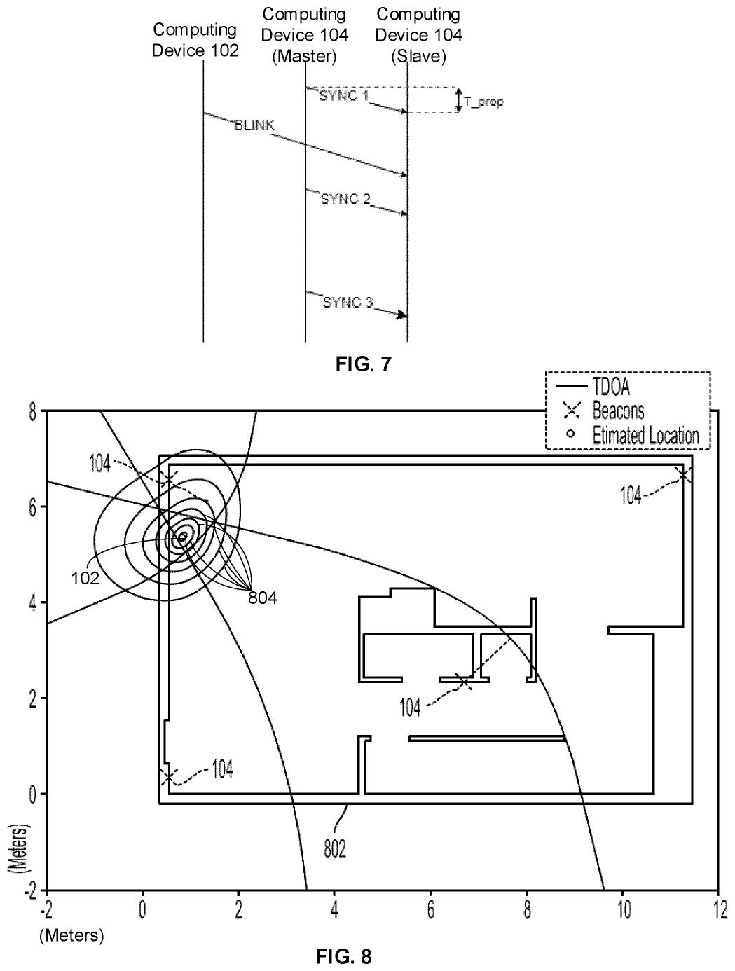

In one embodiment shown in FIG. 7, a linear interpolation may be performed as follows, where:
blinkₘₐₛₜₑᵣ is the timestamp of the reception of the message from one computing device 102 at a slave, according to the master's clock.
blinkₛₗₐᵥₑ is the timestamp of the reception of the message from the computing device 102 at a slave, according to the slave's clock.
   sync1ₘₐₛₜₑᵣ is an the time of transmission of the first sync message, according to the master's clock.
sync2ₘₐₛₜₑᵣ is an the time of transmission of the second sync message, according to the master's clock.
sync1ₛₗₐᵥₑ is the time of reception of the first sync message, according to the slave's clock.
sync2ₛₗₐᵥₑ is the time of reception of the second sync message, according to the slave's clock.
tₚᵣₒₚ is the propagation delay between the master and slave.
c is the propagation velocity.

The propagation delay may be calculated using the known position of the computing device 104, or the mean of the measured propagation delay using the calibration procedure outlined above may be used, where:

$${t}_{p r o p} = \frac{‖ A 1 - A 2 ‖}{c} = \frac{\sqrt{( {x}_{A 1} - {x}_{A 2} ) 2 + ( {y}_{A 1} - {y}_{A 2} ) 2 + ( {z}_{A 1} - {z}_{A 2} ) 2 )}}{c}$$

A1 is a vector of the (x, y, z) coordinates of a first computing device 104.
A2 is a vector of the (x, y, z) coordinates of a second computing device 104.

The master timestamps are then adjusted to account for the propagation delay:

sync1ₘₐₛₜₑᵣ=sync1ₘₐₛₜₑᵣ+tₚᵣₒₚ

sync2ₘₐₛₜₑᵣ=sync2ₘₐₛₜₑᵣ+tₚᵣₒₚ

The timestamp of blinkₛₗₐᵥₑ is then translated so it is referenced to the master's clock:

$${blink}_{master} = \frac{sync {2}_{master} - sync {1}_{master}}{sync {2}_{slave} - sync {1}_{slave}} * ( {blink}_{slave} - sync {1}_{slave} ) + sync {1}_{master}$$

Once the messages received from devices 102 are synchronized to each master, all messages for a single localization may be collected. Messages may be collected on a best effort basis with a short timer which may be reset after each new message is received. When this timer expires, all messages that have been received may be sent for localization. Choosing the timeout length may be a trade-off between network latency and accuracy.

In one preferred embodiment, a collector timeout length may be dynamically adjusted for each facility, region in a facility or for every computing device 102. Given an arbitrary probability distribution of when messages are expected to be received, for example with a normal distribution for a target facility, one may expect messages to take a mean of 1 s, with a standard deviation of 100 ms. This may be used to calculate the minimum timeout length that may result in receiving the target percentage of messages. In the example presented above, if the target was to receive 99.86% of messages, the timeout may be set to 1.3 s. The estimation of the probability distribution may be performed dynamically as parameters change and the communication network 108 of FIG. 1 may become more congested as a result. It should be appreciated that the same technique may be applied for any arbitrary probability distribution, and that the probability distribution may be calculated globally, for each venue, for each region in a venue or for each computing device 102.

It should be appreciated that any suitable localization method may be used in accordance with aspects of the present disclosure, such as minimized mean squared error (MMSE) and particle filter (PF). A TDOA measurement model may be defined as follows:
Computing device 102's position as T(x, y, z)
Computing device 104₁'s position as A₁(x, y, z)
Computing device 104ᵢ's position as Aᵢ(x, y, z)
Propagation Velocity as c

The TDOA between Anchor₁ and Computing device 104ᵢ may be calculated as:

$${TDOA}_{i} = \frac{‖ {A}_{1} - T ‖ - ‖ {A}_{i} - T ‖}{c}$$

Where ∥ . . . ∥ is the Euclidean distance, defined as:

∥ . . . ∥=\sqrt{(x−xᵢ)²+(y−yᵢ)²+(z−zᵢ)²}

The measured TDOA for anchor pair i is:

measᵢ=Timestampᵢ−Timestampᵢ

The error between the calculated TDOA and the measured TDOA is:

eᵢ=TDOAᵢ−measᵢ

To calculate the position using MSE, a cost function may be derived. Using the error function, the mean squared error between all measurements for an arbitrary location of a computing device 102 may be defined as:

$$M S E = \frac{1}{n} {\sum }_{i = 2}^{n} {( {e}_{i} )}^{2}$$

To start, an estimated location may be generated. The MSE cost function is then minimized, in practice a Nelder-Mead Simplex has proven to be effective. In some cases, a local minima may exist and a global optimization routine may need to be performed. An example localization within an environment 802 using MSE is shown in FIG. 8, where the contours 804 show the minimum area position of the estimated location of computing device 102.

MSE has the advantages that it is fast and simple to implement. Since the calculated position may depend on the obtained measurements, it is a useful debugging tool. To reduce noise signals from the calculated position, additional filtering after the position has been calculated (for example with a Kalman Filter) may be carried out. It should be appreciated that other suitable method other than MSE may be used in accordance with aspects of the present disclosure.

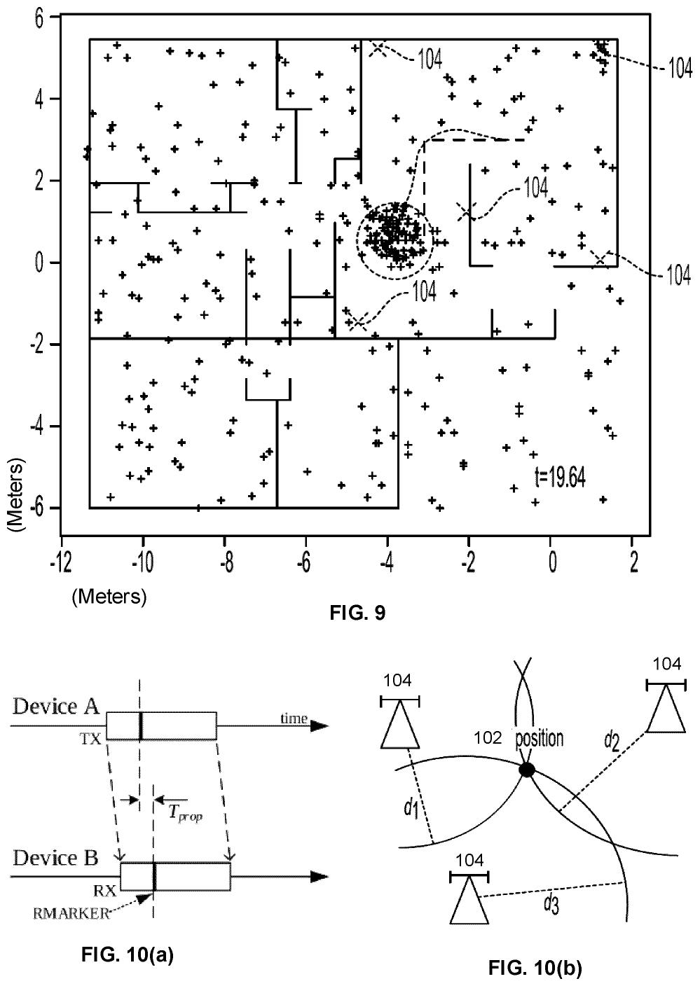

For example, a Particle Filter may have advantages over the MSE approach as it may incorporate additional sources of information, such as expected movement patterns and a floor plan of a facility. Expected movement patterns may include short term historical information such as previous position, previous velocity, and/or previous acceleration, or longer term historical information such as common behavior patterns given examples of similar users or map regions. As shown in FIG. 9, a particle filter may estimate the position of a computing device 102 using a large number of particles. Each particle represents a possible position of the computing device 102. The particles may be weighted depending on the measurements received and in relation to the last position estimate. On every iteration of the filter:
1. The particles may be dispersed (their positions may be updated according to the estimated motion of the person plus some randomness).
2. They may be re-weighted depending on the path they took from their previous position (e.g., whether it is through a wall) and the measurement that was received.
3. The position estimate and confidence interval may be calculated based on the weighted mean and variance of the particles.
4. The best particles may be selected based at least on their weight and new particles are generated around them.

A small subset of the particles may be dispersed randomly throughout an environment to prevent degeneracy of the state estimate. This may occur if several erroneous measurements are received, or the transmitter is “teleported” to a new location (e.g., is turned off and moved, or multiple transmissions fail) and the prior state information is no longer valid. In one embodiment, the random particles may be dispersed uniformly throughout the environment. In one preferred embodiment, the random particles may be distributed around the most likely location based on which computing devices 104 received the transmission from computing device 102. The most likely location may be defined as the mean position of all computing devices 102 that received the transmission plus some random noise, the most probable location based on signal strength, or other convex reliable metric.

In one embodiment, for the senior care space, TDOA may be selected as being the most suitable localization method, but there are many other suitable methods. The propagation delay between a transmission and a reception may be used to determine the range between a transmitter and receiver. Given a minimum of 3 ranges, a 2D position may be determined, or 4 for a 3D position. The transmitter and receiver may be time synchronized. FIG. 10(a) shows an example TOA range, where the propagation delay may be determined by subtracting the transmission time from the reception time. Once the propagation time is known the range is: range=Tₚᵣₒₚ*Vₚᵣₒₚ. For the case of UWB, Vₚᵣₒₚ=c=299792458 (speed of light) m/s. Once a sufficient number of ranges have been collected, a localization may be performed as shown in FIG. 10(b), where the position of a computing device 102 may be obtained by tri-lateration (i.e., N=3) using the minimum number of computing devices 104.

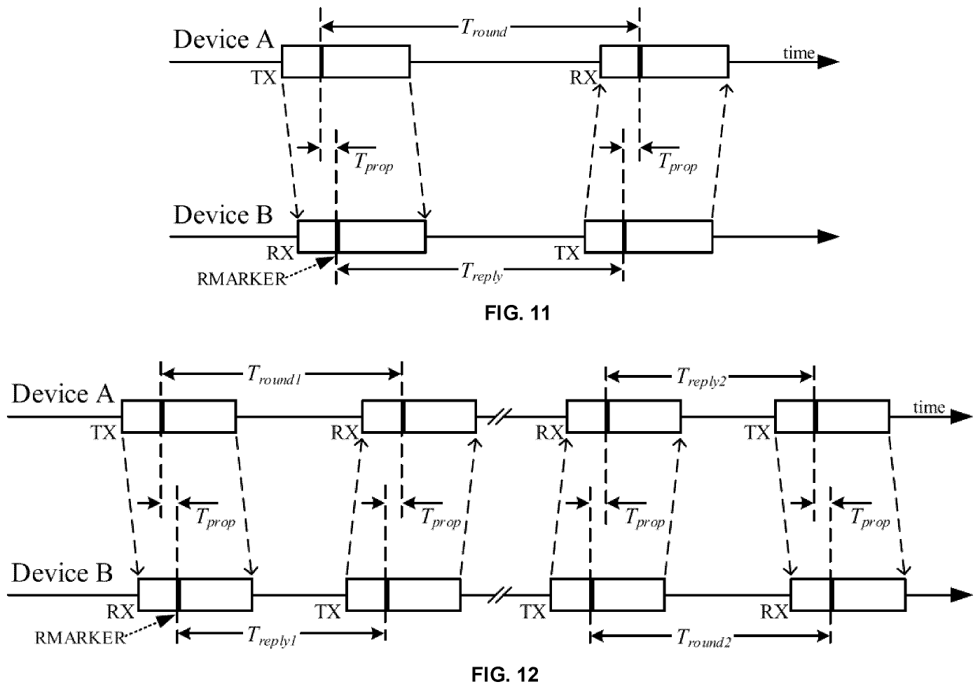

In another embodiment, TWR enables range based algorithms to be used without requiring the transmitter and receiver to be time synchronized. Instead of measuring the propagation delay, the total time elapsed from transmitting a message and receiving a message back is measured, an example exchange is shown in FIG. 11. The processing delay on the receiver is known and is subtracted from the measurement, which leaves 2× propagation delay. However, the processing delay may be measured with the receiver's clock, which may invariably be slightly different than the transmitter's clock. If the processing delay is long, this drift may cause significant error in the measurement. For UWB and other RF signals, the propagation velocity is 3e8 m/s, so 1 ns of error is 30 cm. In addition, this method may require a transmission and a reception for each range to be performed. As discussed above, in one embodiment, a localization may require a minimum 3 ranges for 2D localization or 4 for 3D. This large number of transmissions and receptions may consume significant power and use a large portion of airtime, negatively affecting battery life and reducing the number of devices that may be supported.

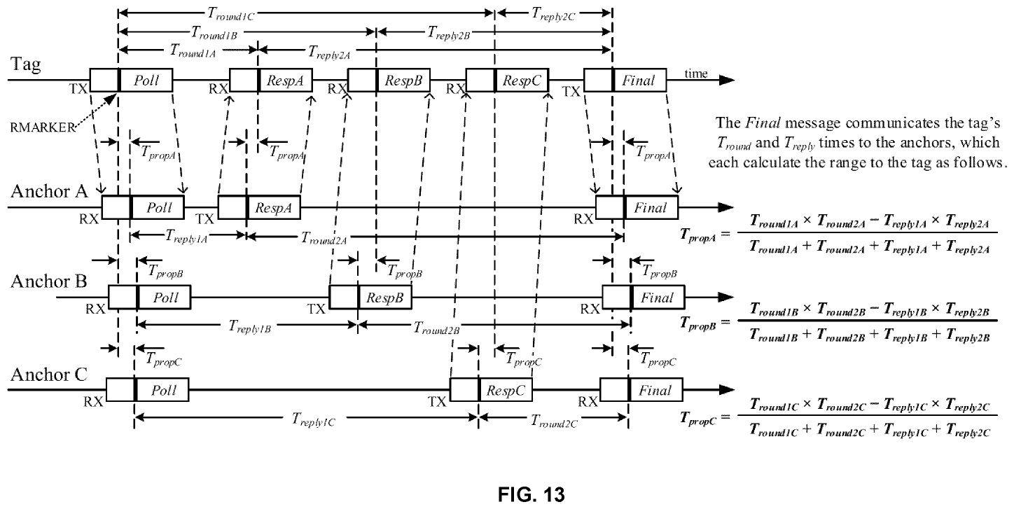

Double sided two way ranging (DS-TWR) is a slight modification on the TWR algorithm, as shown in FIG. 12. The skew caused by the clock drift may be reduced since there is processing time on both transmitter and receiver units. A more complete explanation is presented in D. Neirynck et al., “An alternative double-sided two-way ranging method” in 2016 13^{th} workshop on positioning, navigation and communications (WPNC), pages 1-4, which is incorporated herein in its entirety. This method increases the accuracy significantly, while additional messages are required. If the processing delay on both transmitter and receiver units are equivalent, the clock skew may cancel out completely (assuming the skew does not change significantly over time). In one implementation as shown in FIG. 13, the number of messages may be reduced by starting with a broadcast to all computing devices 104 within range, receiving each computing device 104 reply in turn, then sending a final transmission with all the results.

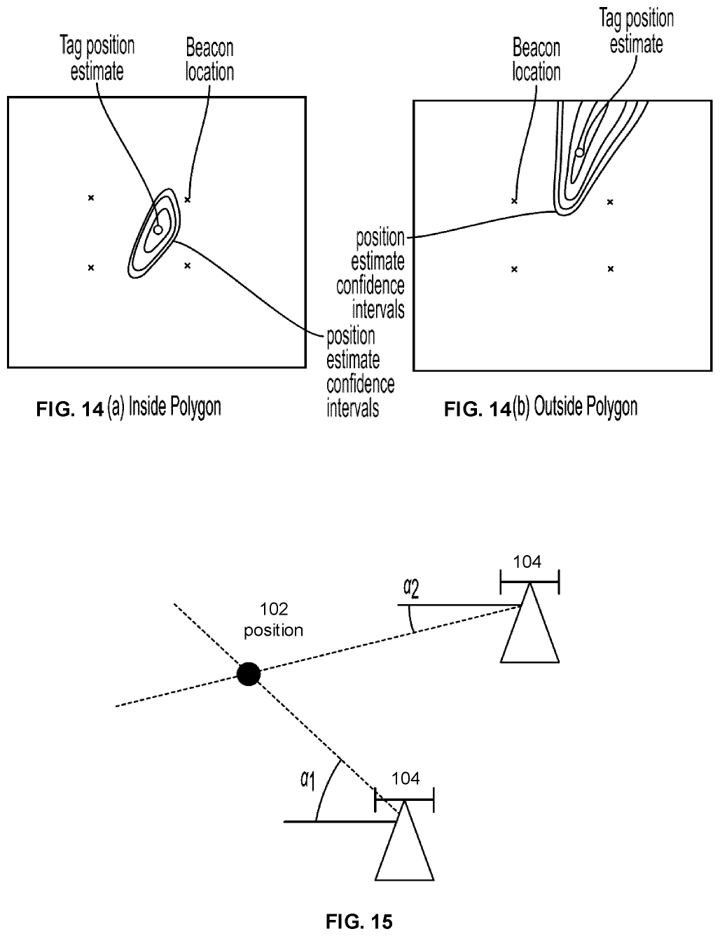

In one embodiment, a transmitter such as computing device 102 may be only required to transmit once and may not need to receive any messages, thereby consuming very little power and allowing a high number of transmitters to be supported by system 100 of FIG. 1. The receiving nodes may share a common time base, so some form of wired or wireless synchronization may be utilized. Poor synchronization between the nodes decreases the system accuracy. Additionally, the accuracy degrades very quickly when the transmitter is outside of a polygon formed by the receiving nodes. FIGS. 14(a) and 14(b) compare a localization inside and outside of a polygon, respectively. In a preferred embodiment, receiving nodes may be placed such that they entirely enclose the space where localization is to be performed. In certain cases this may not be possible due to physical restrictions. In other cases accurate localization may be required near the edge of the polygon.

In one embodiment, the out of polygon error may be minimized by augmenting the TDOA measurements with a single TWR measurement. By using only a single TWR measurement the power consumption of computing device 102 remains reasonable. The choice of computing device 104 to perform the TWR with is critical. Due to the geometry of the error, the layout of computing devices 104 and the expected ranging error, the choice of computing device 104 to perform TWR with may greatly affect the localization error. Since the time between computing device 102 sending a transmission, receiving a response and sending out a final transmission must be minimized for both accuracy (due to relative clock drift) and battery (reduce time not in sleep mode), it may be advantageous to pre-select the computing device 104 that is responsible for responding to the TWR. This may be done by selecting the device 104 that will minimize the error at the next timestamp from a list of devices 104 that are likely to receive a transmission from the target device 102. The list of devices 104 may be generated based on the distance from the estimated location of the target device 102 at the previous timestamp and the list of devices 104 that received a message from the target device 102 at the previous timestamp. The estimated error may be calculated using the measured TDOA variation at the previous timestamp and the expected range error based on calibration data, the previous received message and/or the location of the device 102 at the previous timestamp and the construction materials of the environment. For each potential device 104, the expected error may be calculated as if it was the selected responsible device. The one with the minimum error may then be selected to respond to the target device 102. In another embodiment, additional metrics may be added to prevent the responsible device 104 from updating too frequently.

In another embodiment, the TDOA measurements may be augmented with Received Signal Strength Indicator (RSSI) information. Generally, the signal strength of a message may be high when the transmitter is close to the receiver and there are few obstructions. The signal strength of a message may be decreased when the transmitter is close to the receiver and there are obstructions, or when the transmitter is far from the receiver. This does not require any additional transmissions from computing device 102 and therefore does not negatively impact the battery life. A model that is configured to take and RSSI and distance values and return a likelihood may be generated from ground truth data composed of a number of transmitters at known locations. The data and hence resulting model may be static, or may be re-collected dynamically whenever the environment is expected to have changed, or on a regular basis. The data and hence resulting model may be collected for a single venue, a single class of venue, or for throughout a grid in a target venue down to an arbitrarily small grid size. In one embodiment, this model may be a two dimensional spline that interpolates the gathered (RSSI, Distance) data. It should be appreciated that there are a number of ways to generate such a model. In one embodiment, if a Particle Filter is used for localization the calculated likelihood may be used to update the particles' weight, for example: w=a*w_rssi+(1−a)*w_tdoa, where w is the final weight for each particle, a is the ratio between the TDOA measurement and the RSSI measurement, w_rssi is the weight (likelihood) of the RSSI measurement and w_tdoa is the weight (likelihood) of the TDOA measurement. Since the RSSI measurements are very stable (e.g., the accuracy is generally worse than a meter, but never worse than 10 meters), augmenting the TDOA measurements with the RSSI measurements may prevent any large position errors that could be possible with TDOA.

Referring to FIG. 15, by using multiple antennas on a single computing device 104, the angle of an arriving signal may be determined. Taking two antennas for example, the angle of arrival may be determined in a 180 degree field. Signals from behind may not be distinguished from signals in front, and the accuracy degrades as the angle approaches 0 and 180 degrees since the path difference diminishes. In one embodiment, only two computing devices 104 may be required for a 2D localization to be performed. Similar to TDOA, each computing device 102 may only need to emit a single transmission to localize, allowing the system 100 shown in FIG. 1 to support a high number of computing devices 102 with prolonged battery life. In one embodiment, more antennas and measurements may be required if the position of computing device 102 is to be determined in 3D.

The antenna spacing and layout is very important for Angle of Arrival (AOA) applications. The AOA may be calculated either based on the differential time of arrival at each antenna and the propagation velocity, or the differential phase of arrival and the wavelength of the signal. The maximum spacing of the antenna may be constrained by the physical dimensions of the beacon. This may put a limit on the accuracy of the timestamping. Often, the noise floor of the timestamping may be greater than the desired precision, so a purely timestamp based method may not yield useful results. Measuring the carrier frequency phase difference may be more accurate for smaller antenna spacing. For example, to maximize phase difference resolution without any angle ambiguities, the antennas may be spaced ½ wavelength apart. For UWB channel 5 (6.489 GHz), one example spacing may be 2.31 cm. Antenna spacing greater than ½ wavelength may have a higher resolution, but a given phase difference may be caused by multiple different transmission angles. Therefore, an additional mechanism may be needed to reduce these angle ambiguities, such as a time difference measurement at each antenna, or prior knowledge about the transmission location. Additionally, all AOA methods may suffer from reduced precision when the transmission source is near to being collinear with the antennas due to changes in angle only causing a small change in path difference. Calibration procedures may be used to compensate for any non-linearity or other errors in the conversion from phase difference and/or time differential to angle.

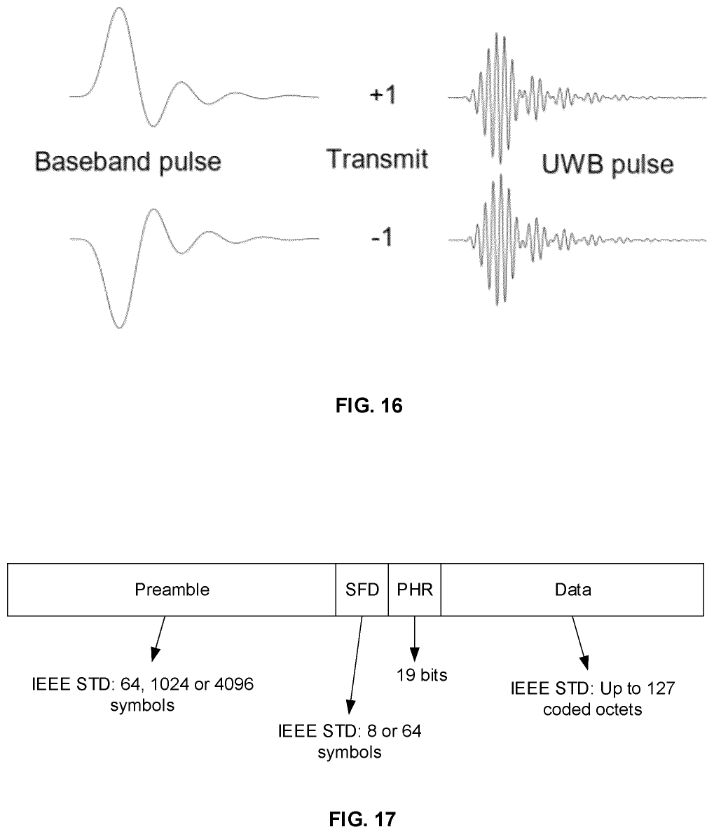

In order to perform accurate localization, the reception of a signal may be accurately timestamped, and delayed signals that are caused by reflections may be eliminated. UWB may be designed to address both issues. UWB is a carrier based impulse radio. As shown in FIG. 16, a baseband impulse may be generated at a chipping rate of 499.2 MHz, and then mixed with a channel dependent carrier frequency. The baseband impulse may either be +1 or −1.

A UWB frame may be made up of the sections shown in FIG. 17. The preamble and the SFD (Start Frame Delimiter) may be the only sections used for ranging. The preamble may include a known sequence selected from an available set of perfect ternary sequences that is transmitted repeatedly. A perfect ternary sequence may include a sequence with perfect periodic autocorrelation. Autocorrelation may be the correlation of the signal with a delayed copy of itself. Perfect autocorrelation means the correlation is at a maximum when the delay is zero, and at a minimum at all other times. An example perfect ternary sequence 31 units long is: −0000+0−0+++0+−000+−+++00−+0−00. UWB may use the signal phase (+1/−1) and position (present or not present) to represent the ternary signals −1, 0, 1 (Binary Phase Shift Keying/Burst Position Modulation (BPSK/BPM)). Since the transmitted signal may be known, all other variations in the received signal may be due to noise and environmental effects such as reflections. This allows the receiver to build a model of the Channel Impulse Response (CIR). When the preamble is complete, the Start Frame Delimiter (SFD) shown in FIG. 17 may mark the moment to be timestamped.

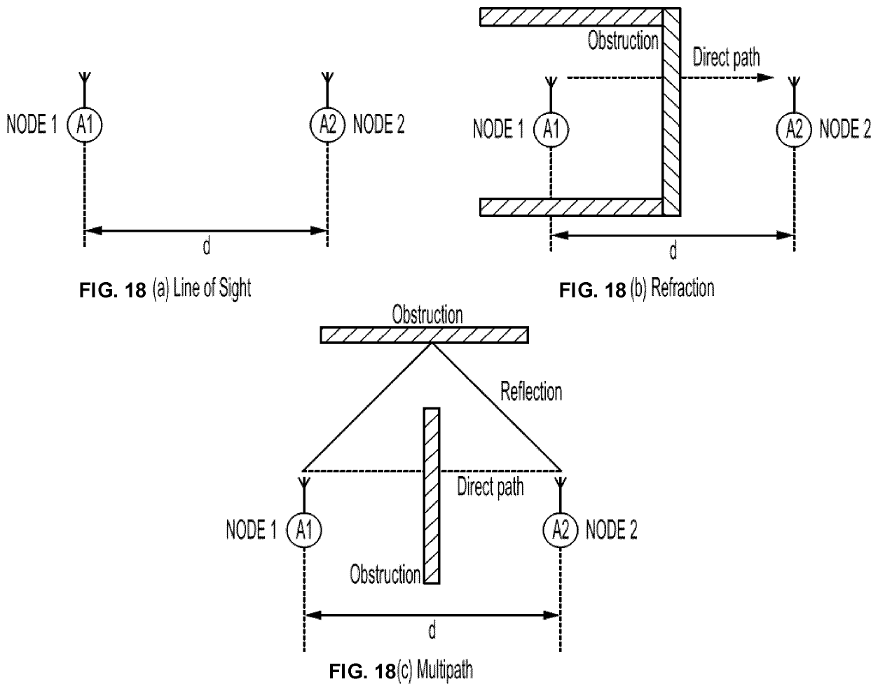

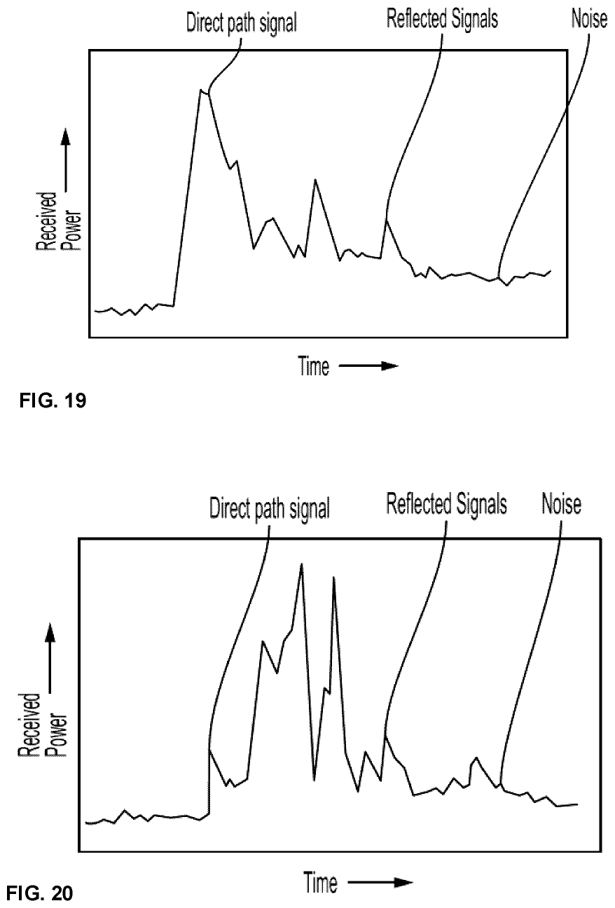

The first path (direct path) of the signal may be determined from the CIR. The first path may be the earliest possible signal received. It is possible that the first path is below the noise threshold and may not be accurately determined. All other signals in the CIR may be caused by reflections or refractions in the environment, both of which delay the signal. FIGS. 18(a), 18(b) and 18(c) show a few simple scenarios, a typical indoor environment may be composed of many of these scenarios superimposed. A CIR for an indoor Line Of Sight (LOS) measurement with a clear first path and reflections is shown in FIG. 19, a CIR for an indoor Non Line Of Sight (NLOS) measurement is shown in FIG. 20, the first path is attenuated but still visible. A setup such as FIG. 18(c) would cause this behavior. Specifically, FIGS. 19 and 20 show example CIR data for two different scenarios, where FIG. 19 shows a reasonable quality line of sight signal, as would be expected in a situation akin to FIG. 18(a). The earliest signal received may also be the largest in magnitude. Later signals may include reflections that are attenuated due to the longer path distance. FIG. 20 shows an NLOS scenario as would be encountered in FIG. 18(c). The earliest signal may be attenuated compared to the time delayed reflections. In certain cases, there may be errors in the received timestamp. This may be caused by noise in the environment, attenuation of the first path signal, and/or strong reflections.

In one embodiment, the timestamp errors may be corrected with information inferred from the CIR, knowledge of the environment, previously gathered information and/or agreement with other received measurements. A confidence in the timestamp may also be calculated in a similar manner. For example, as discussed earlier, a signal with multiple reflections stronger than the first path, potential ambiguous early paths near the noise floor and low overall signal power are more likely to be erroneous and so should have less impact on the position estimate. Exact parameters for how to weight the extracted features to minimize error may be determined using a known location dataset to determine the parameters that minimize the error.

In one embodiment, the measurements may also be corrected using the initial position estimate or the estimate at the previous timestamp and knowledge of how the building materials affect the signal. For example, when a signal passes through a wall, it may be attenuated and delayed based on the properties of the material such as the thickness, angle of penetration, attenuation constant, and refractive index. With an estimate of the transmitter's location and knowledge of the receiver's location, the obstructions that the signal may have penetrated may be determined. Example corrections may include either adjusting the timestamp of the signal to account for the delay, or discarding the measurement entirely because it was determined that the first path should have been completely attenuated. These corrected measurements may then be used to determine a more accurate position estimate.

In one embodiment, computing device 104 may be configured to transmit messages that mimic the messages computing devices 102 transmit. Since the location of computing devices 104 is known, this may allow the localization system 100 of FIG. 1 to be tested with a number of known location devices to measure expected performance, calibrate any constant position errors, characterize environmental parameters such as construction materials, and identify possible problem locations.

The following will describe how the disparate pieces of the above disclosure may be combined into a unique system to perform accurate indoor localization in complex indoor environments. First a number of fixed position computing devices 104 and installed throughout the area to be covered. The computing devices 104 are capable of performing time of flight measurements to a sufficient degree of accuracy. The computing devices 104 may perform a calibration whereby they perform TWR multiple times with all possible peers. The calibration data may be used to select a subset of the computing devices 104 as master devices that will periodically send out clock synchronizations messages. The set of master devices may be selected to optimally cover the area with the best possible accuracy. The synchronization messages may be dynamically adjusted to increase accuracy and reliability. A number of mobile unknown location computing devices 102 are within the facility and periodically transmit updates depending on their state of motion. The computing devices 102 may be required to be wearable by a human or mounted to an asset so they must be small and have a battery life of several months. Using the time of reception of a transmission from one of the plurality of computing devices 102 by a subset of the plurality of computing devices 104 multiple TDOA measurements may be calculated. The TDOA measurements may be augmented by additional measurements such as a TWR from a pre-selected computing device 104, RSSI measurements from the subset of the plurality of computing devices 104, sensors onboard the computing device 102 such as an IMU, or sensors onboard both computing devices such as barometers. To combine the various measurement sources a filter such as a Kalman filter (or variant such as extended Kalman filter (EKF) or unscented Kalman filter (UKF)) or a particle filter may be used. The measurement data may be augmented with environment data that contains information about walls or other obstructions that would impede movement. The environment data may also contain information relating to the effect on the signal quality such that errors in the signal may be estimated and/or corrected. The measurement data may also be augmented with historical data, both short term (e.g., from the last update), or long term (e.g., trends over the course of days or weeks). The resulting data may be used to improve operational efficiency, or depending on the application, for example a healthcare facility, improve health outcomes or make predictive behavior analytics.

The above description of the disclosure is provided to enable a person skilled in the art to make or use the disclosure. Various modifications to the disclosure will be readily apparent to those skilled in the art, and the common principles defined herein may be applied to other variations without departing from the spirit or scope of the disclosure.

Furthermore, although elements of the described aspects and/or embodiments may be described or claimed in the singular, the plural is contemplated unless limitation to the singular is explicitly stated. Additionally, all or a portion of any aspect and/or embodiment may be utilized with all or a portion of any other aspect and/or embodiment, unless stated otherwise. Thus, the disclosure is not to be limited to the examples and designs described herein but is to be accorded the widest scope consistent with the principles and novel features disclosed herein.

1. A system, comprising:
a plurality of first computing devices, wherein each first computing device is wearable by a user or is affixed to an asset within an environment and configured to generate and transmit a message; and
a plurality of second computing devices installed within the environment, wherein a portion of the plurality of second computing devices are configured to detect at least one of the plurality of first computing devices, receive messages transmitted by a detected first computing device, and timestamp the messages before transmitting the messages to a computing server system,
wherein the computing server system is configured to determine at least a location of the detected first computing device based at least upon the messages received from the portion of the plurality of second computing devices.

2. The system of claim 1, wherein the computing server system is configured to obtain information relating to an environment in which the plurality of first and second computing devices are deployed, wherein the information comprises parameters indicating at least one of: locations of walls or physical obstructions of the environment that would impede movement wherein computing server system is configured to determine the location of the detected first computing device using at least the messages received from the portion of the plurality of second computing devices and the information.

3. The system of claim 2, wherein the computing server system is configured to determine the location of the detected first computing device using at least time difference of arrival (TDOA) measurements from the portion of the plurality of second computing devices, and a single two way ranging (TWR) measurement from one of the plurality of second computing devices.

4. The system of claim 3, wherein the one of the plurality of second computing devices that performs the TWR measurement is pre-selected based at least on an estimated position of the detected first computing device, an estimate of which ones of the plurality of second computing devices receive a next message from the detected first computing device, and a method for minimizing a measurement uncertainty.

5. The system of claim 4, wherein the one of the plurality of second computing devices that performs the TWR measurement for the next message is determined based at least on measurements received from the detected first computing device at a previous message, and a set of the plurality of second computing devices that are likely to receive from the detected first computing device at the next message, wherein, for each of the set of the plurality of second computing devices, an expected position error is calculated, and measurements from the previous message are used to estimate an expected variance of the TWR measurement, wherein the method is configured to select one of the plurality of the second computing devices with the minimum expected error and determine whether to replace a previously selected second computing device for performing the TWR measurement.

6. The system of claim 2, wherein the computing server system is configured to determine the location of the detected first computing device using at least time difference of arrival (TDOA) measurements from at least one of the plurality of second computing devices, received signal strength indicator (RSSI) measurements from the plurality of second computing devices.

7. The system of claim 1, wherein the plurality of second computing devices are calibrated to measure a propagation delay between itself and every other second computing device within a communication range using at least a single two way ranging (TWR) or double sided TWR (DS-TWR) methods.

8. The system of claim 7, wherein the plurality of second computing devices are calibrated to estimate a quality of a communication channel between itself and every other second computing device within the communication range by measuring the propagation delay multiple times and calculating a standard deviation, fitting an arbitrary probability distribution, or other approximation from discrete data.

9. The system of claim 7, wherein the plurality of second computing devices are calibrated once during system installation, periodically, continuously, or as required when changes are detected in the system.

10. The system of claim 1, wherein the plurality of second computing devices comprise master devices and slave devices.

11. The system of claim 10, wherein each master device is selected from the plurality of second computing devices based at least on: maximizing an area formed by each cluster having a master device and multiple slave devices, having the best quality communication between each master device and the multiple slave devices, each second computing device has at least one master device, and minimizing a total number of required master devices.

12. The system of claim 10, wherein each master device is configured to periodically transmit a synchronization message, wherein the synchronization message includes at least information relating to a local clock of each master device at the time of transmission.

13. The system of claim 12, wherein each slave device is configured to receive the synchronization message and timestamp the synchronization messages upon reception.

14. The system of claim 1, wherein each of the plurality of second computing devices is configured to determine a channel impulse response (CIR) for each signal received from each of the plurality of first computing devices, and the computing server system is configured to determine the location of the detected first computing device based at least on the CIR of each signal.

15. The system of claim 1, wherein the system is deployed within a cloud-based network communication system.

16. A method, comprising:
generating and transmitting one or more messages from each of a plurality of first computing devices wearable by a user or is affixed to an asset within an environment;
installing a plurality of second computing devices within the environment;
detecting, by a portion of the plurality of second computing devices, at least one of the plurality of first computing devices;
receiving, by the portion of the plurality of second computing devices, messages transmitted by a detected first computing device;
timestamping the messages by the portion of the plurality of second computing devices before transmitting the messages to a computing server system; and
determining, by the computing server system, at least a location of the detected first computing device based at least upon the messages received from the portion of the plurality of second computing devices.

17. A non-transitory computer-readable medium storing computer codes executable by one or more processors of a system to:
generate and transmit one or more messages from each of a plurality of first computing devices wearable by a user or is affixed to an asset within an environment, wherein a plurality of second computing devices are installed within the environment;
detect, by a portion of the plurality of second computing devices, at least one of the plurality of first computing devices;
receive, by the portion of the plurality of second computing devices, messages transmitted by a detected first computing device;
timestamp the messages by the portion of the plurality of second computing devices before transmitting the messages to a computing server system; and
determine, by the computing server system, at least a location of the detected first computing device based at least upon the messages received from the portion of the plurality of second computing devices.
